(19) United States
(12) Patent Application Publication
(10) Pub. No.: US 2026/0115000 A1
(43) Pub. Date: Apr. 30, 2026

(54) IMPLANTABLE MEDICAL DEVICE WITH A CIRCUIT SUBSTRATE COUPLED TO A FLUIDIC MANIFOLD

(71) Applicant: Boston Scientific Scimed, Inc., Maple Grove, MN (US)

(72) Inventors: Matthew William Couri, Plymouth, MN (US); John Anders Bostrom, Saint Paul, MN (US); Trey Henry Achterhoff, Arden Hills, MN (US); Derek John Boettger, Woodbury, MN (US); Henry Koon, Saint Paul, MN (US); Paul John Gindele, Buffalo, MN (US)

(21) Appl. No.: 19/370,003

(22) Filed: Oct. 27, 2025

Related U.S. Application Data
(60) Provisional application No. 63/714,561, filed on Oct. 31, 2024.

(51) Int. Cl. A61F 2/26; A61F 2/00; A61F 2/48

(52) U.S. Cl. CPC A61F 2/26; A61F 2/0027; A61F 2/482; A61F 2220/0033; A61F 2220/0041; A61F 2220/005

(57) ABSTRACT

An implantable medical device may include an inflatable member. An implantable medical device may include a fluid reservoir. An implantable medical device may include an electronic pump device configured to transfer fluid between the inflatable member and the fluid reservoir. The electronic pump device includes a housing. The housing includes a fluidic manifold including a plurality of fluidic components. The manifold includes an inside edge and a shelf that extends from the inside edge. The housing includes a circuit substrate contacting the shelf. The circuit substrate includes a hole. The housing includes a coupling member that couples the circuit substrate to the shelf. The coupling member extends through the hole.

### CROSS-REFERENCE TO RELATED APPLICATION

This application claims priority to U.S. Provisional Patent Application No. 63/714,561, filed on Oct. 31, 2024, entitled “IMPLANTABLE MEDICAL DEVICE WITH A CIRCUIT SUBSTRATE COUPLED TO A FLUIDIC MANIFOLD”, the disclosure of which is incorporated by reference herein in its entirety.

### TECHNICAL FIELD

This disclosure relates generally to an implantable medical device with an electronic pump device having a circuit substrate coupled to a fluidic manifold.

### BACKGROUND

Some inflatable medical devices have a manual pump, which, when operated by a user, causes a transfer of fluid between a fluid reservoir and an inflatable member.

### SUMMARY

In some aspects, the techniques described herein relate to an implantable medical device including: an inflatable member; a fluid reservoir; and an electronic pump device configured to transfer fluid between the inflatable member and the fluid reservoir, the electronic pump device including a housing, the housing including: a fluidic manifold including a plurality of fluidic components, the manifold including an inside edge and a shelf that extends from the inside edge; a circuit substrate contacting the shelf, the circuit substrate including a hole; and a coupling member that couples the circuit substrate to the shelf, the coupling member extending through the hole.

In some aspects, the techniques described herein relate to a method including: positioning a circuit board on a shelf of a fluidic manifold of an electronic pump device of an implantable medical device; and coupling the circuit board to the shelf using a coupling member.

In some aspects, the techniques described herein relate to an implantable medical device including: an inflatable member; a fluid reservoir; and an electronic pump device configured to transfer fluid between the inflatable member and the fluid reservoir, the electronic pump device including a housing, the housing including: a fluidic manifold including a plurality of fluidic components, the manifold including an inside edge and a shelf that extends from the inside edge; a circuit substrate contacting the shelf, the circuit substrate including a hole; and a coupling member that couples the circuit substrate to the shelf, the coupling member extending through the hole.

In some aspects, the techniques described herein relate to a method including: positioning a circuit board on a shelf of a fluidic manifold of an electronic pump device of an implantable medical device; and coupling the circuit board to the shelf using a coupling member.

In some aspects, the techniques described herein relate to an implantable medical device including: an inflatable member; a fluid reservoir; and an electronic pump device configured to transfer fluid between the inflatable member and the fluid reservoir, the electronic pump device including a housing, the housing including: a fluidic manifold including one or more pumps, one or more valves, or one or more pressure sensors, the fluidic manifold including a frame with an inside edge, the fluidic manifold including a shelf that extends from the inside edge; a circuit substrate contacting the shelf, the circuit substrate including a plurality of electronic components, the circuit substrate including a hole; and a coupling member that couples the circuit substrate to the shelf, the coupling member extending through the hole.

### BRIEF DESCRIPTION OF THE DRAWINGS

FIG. 1A illustrates an implantable medical device with an electronic pump device having a circuit board coupled to a fluidic manifold according to an aspect.

FIG. 1B illustrates a fluidic manifold with a shelf according to an aspect.

FIG. 1C illustrates a circuit substrate coupled to the fluidic manifold according to an aspect.

FIG. 1D illustrates a cross-sectional view of a fluidic manifold according to an aspect.

FIG. 1E illustrates an enlarged portion of a cross-sectional view of a fluidic manifold according to an aspect.

FIG. 1F illustrates an exploded view of a housing of an electronic pump device according to an aspect.

FIG. 2A illustrates an example of a post extending from a shelf of a fluidic manifold according to an aspect.

FIG. 2B illustrates an example of a clip member coupled to the post according to an aspect.

FIG. 2C illustrates a cross-sectional view of the fluidic manifold according to an aspect.

FIG. 3A illustrates a circuit substrate coupled to a fluidic manifold using a post and an adhesive material according to an aspect.

FIG. 3B illustrates a post extending from a shelf, a circuit board with a hole that includes a portion of the post, and an adhesive material on the post according to an aspect.

FIG. 4 illustrates a circuit substrate coupled to a fluidic manifold using a post and a ring member according to an aspect.

FIG. 5 illustrates a circuit substrate coupled to a fluidic manifold using a rivet according to an aspect.

FIG. 6 illustrates a circuit substrate coupled to a fluidic manifold using injection molding according to an aspect.

FIG. 7 illustrates a circuit substrate coupled to a fluidic manifold using a threaded fastener according to an aspect.

FIG. 8 illustrates a perspective of an inflatable penile prosthesis according to an aspect.

FIG. 9 illustrates an example of an artificial urinary sphincter device according to an aspect.

FIG. 10 illustrates a flowchart depicting example operations of coupling a circuit substrate to a fluidic manifold of an electronic pump device according to an aspect.

### DETAILED DESCRIPTION

This disclosure relates to an implantable medical device with an electronic pump device configured to automatically transfer fluid between a fluid reservoir and an inflatable member. In some examples, the implantable medical device includes a penile prosthesis with one or more inflatable cylinders. In some examples, the implantable medical device includes a urinary control device with an inflatable cuff. The electronic pump device includes a housing with a fluidic manifold having one or more pumps and one or more valves to facilitate the transfer of fluid between the fluid reservoir and the inflatable member. The fluidic manifold includes a shelf that retains and is coupled to a circuit substrate with a plurality of circuit components (e.g., electronic components). The circuit substrate includes a hole. The electronic pump device includes a coupling member that couples the circuit substrate to the shelf, where the coupling member extends through the hole.

In some examples, the coupling member includes a post that is integral to and extends from the shelf. In some examples, the coupling member includes a clip coupled to the post, where the circuit substrate has a portion disposed between the clip and the shelf. In some examples, the coupling member includes an adhesive material disposed on the post. In some examples, the coupling member includes a ring member coupled to the post, where the circuit substrate has a portion disposed between the ring member and the shelf. In some examples, the coupling member includes a rivet. In some examples, the coupling member includes injection molding. In some examples, the coupling member includes a threaded fastener.

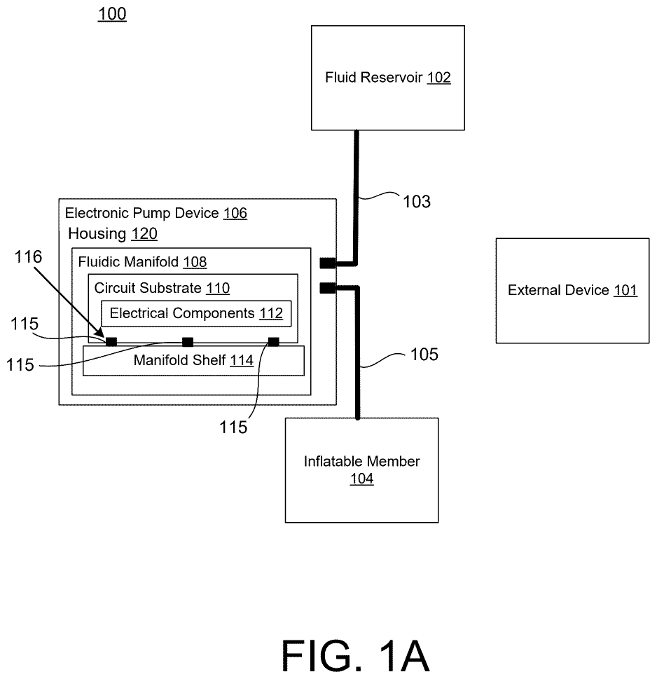

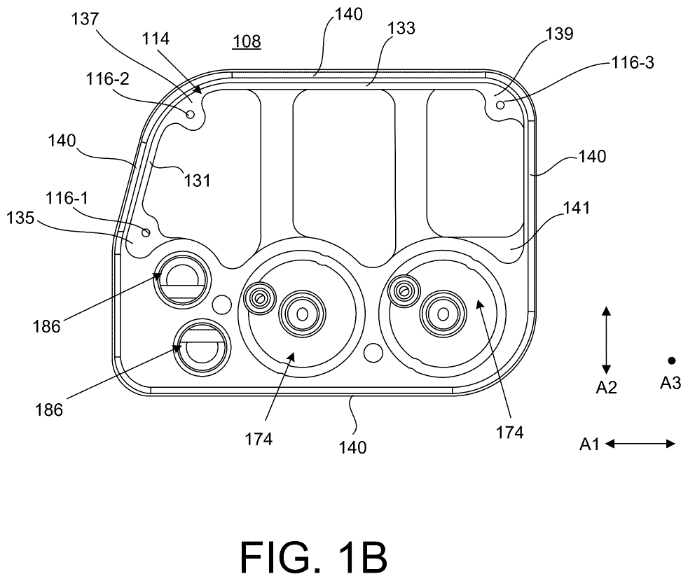

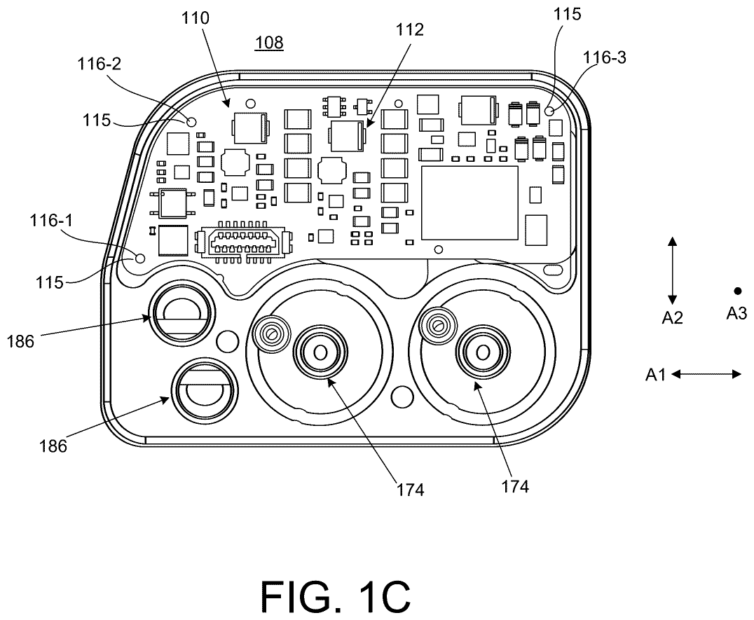

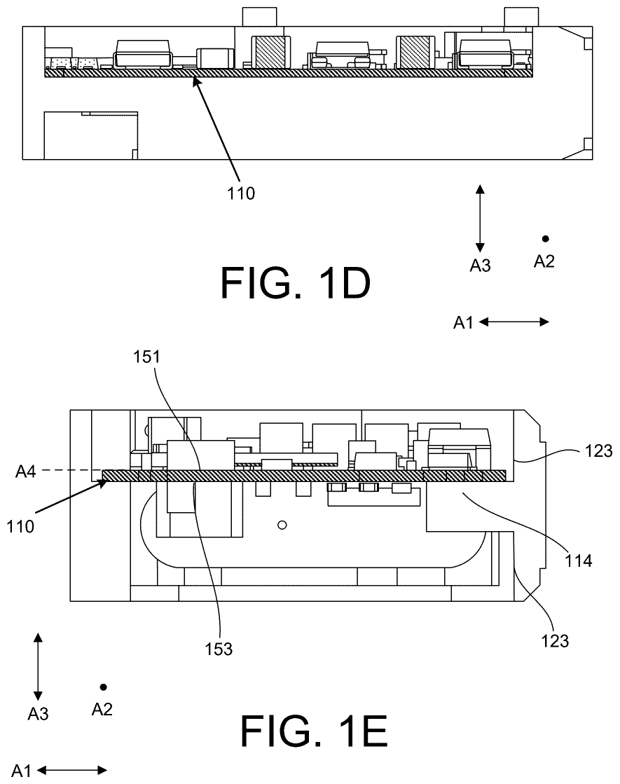

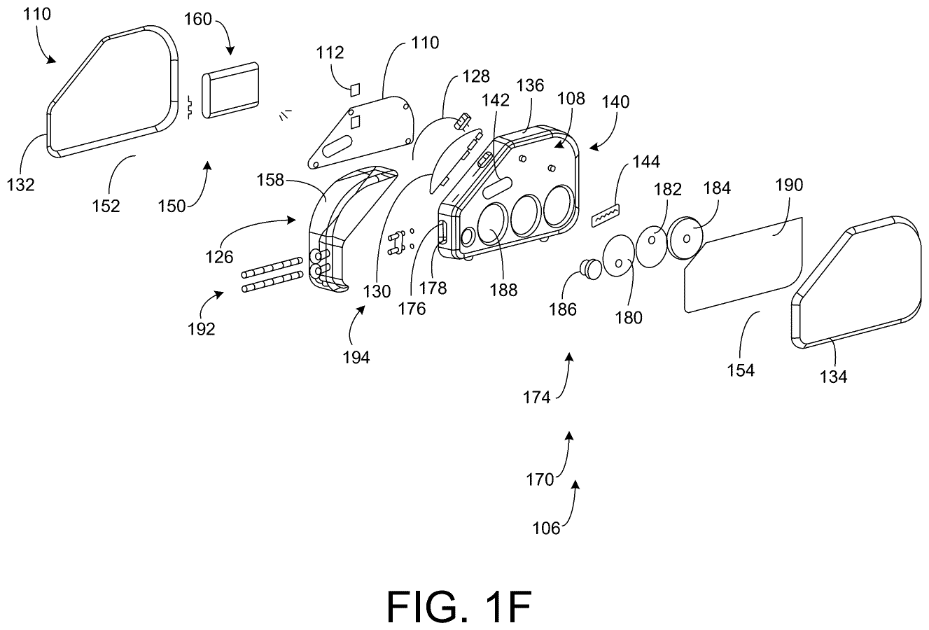

FIGS. 1A-1F illustrates an implantable medical device 100 according to an aspect. In some examples, the implantable medical device 100 is an artificial urinary sphincter device. In some examples, the implantable medical device 100 is an inflatable penile prosthesis. However, the implantable medical device 100 may include any type of medical device that transfers fluid between components of the implantable medical device 100.

The implantable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump device 106 may be implemented in the patient's body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 that can monitor control and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 may include an antenna configured to wirelessly transmit (and receive) wireless signals from an external device 101. The external device 101 may be any type of component that can communicate with the electronic pump device 106. The external device 101 may be a computer, smartphone, tablet, pendant, key fob, etc. A user may use the external device 101 to control the implantable medical device 100. In some examples, the user may use the external device to inflate or deflate the inflatable member 104.

Referring to FIGS. 1A to 1E, the electronic pump device 106 includes a housing 120. The housing 120 includes a fluidic manifold 108 that attaches fluid transfer and pressure regulating components and a circuit substrate 110 with electronic components 112. In some examples, the circuit substrate 110 is a printed circuit board (PCB). The fluidic manifold 108 may include fluidic components 174 such as one or more pumps (e.g., electrically controlled pumps) and one or more valves. The fluidic manifold 108 may include one or more pressure sensors 186. In some examples, the pressure sensors 186 include a first pressure sensor connected to the fluid reservoir 102 to detect a pressure in the fluid reservoir 102, and a second pressure sensor connected to the inflatable member 104 to detect a pressure in the inflatable member 104. The fluidic manifold 108 includes a frame 140 with an inside edge 123. The frame 140 may define a peripheral wall 136 (see FIG. 1F), which is a portion of the outer surface of the electronic pump device 106. The fluidic manifold 108 includes a shelf 114 that extends from the inside edge 123.

The shelf 114 may extend from the inside edge 123. The circuit substrate 110 contacts the shelf 114. The circuit substrate 110 may sit on top of the shelf 114 such that the circuit substrate 110 is positioned within the frame 140. As shown in FIG. 1E, the circuit substrate 110 includes a first surface 151 and a second surface 153 that is opposite to the first surface 151. The first surface 151 is disposed in a plane A4. The thickness of the circuit substrate 110 is defined as the distance between the first surface 151 and the second surface 153 in the direction A3. The direction A3 is orthogonal to the plane A4. The direction A1 is perpendicular to the direction A3 and perpendicular to the direction A2. The direction A2 is perpendicular to the direction A1 and perpendicular to the direction A3.

The circuit substrate 110 includes one or more holes 115. In some examples, the holes 115 extend entirely through the thickness of the circuit substrate 110 in the direction A3. The electronic pump device 106 includes one or more coupling members 116 that couple the circuit substrate 110 to the shelf 114. A coupling member 116 may be a component that can extend into a hole 115. In some examples, the coupling member 116 includes one or more retaining features that hold the circuit substrate 110 on the shelf 114. In some examples, a coupling member 116 includes a post that is integral to and extends from the shelf 114. However, the coupling member 116 may be other types of fasteners as discussed herein. As shown in FIGS. 1A to 1C, the coupling members 116 may include a coupling member 116-1, a coupling member 116-2, and a coupling member 116-3. Each of the coupling member 116-1, the coupling member 116-2, and the coupling member 116-3 may be inserted into a respective hole 115 on the circuit substrate 110.

As shown in FIG. 1B, the shelf 114 may include one or more shelf portions (e.g., also referred to as shoulder portions) that extend from the inside edge 123 of the frame 140 in the directions A1 and A2. The shelf 114 may have a thickness that extends in the direction A3. In some examples, the shelf portions extend from the inside edge 123 in the direction A2 and/or the direction A3 at multiple different lengths. The shelf portions may include a corner portion 135, a corner portion 137, a corner portion 139, and a corner portion 141. The corner portion 135 may define, include, or contact a coupling member 116-1. The corner portion 137 may define, include, or contact a coupling member 116-2. The corner portion 139 may define, include, or contact a coupling member 116-3. The shelf portions may include a connecting portion 131 that extends between the corner portion 135 and the corner portion 137. The shelf portions may include a connecting portion 133 that extends between the corner portion 137 and the corner portion 139.

FIG. 1F illustrates an example of an exploded view of an electronic pump device 106 according to an aspect. The housing 120 includes a first sidewall 132, a second sidewall 134, a peripheral wall 136, and a frame 140. The first sidewall 132, second sidewall 134, and the peripheral wall 136 are hermetically sealed together to form an internal compartment 150 within the housing 120.

The frame 140 is disposed within the internal compartment 150 to form a first partition 152 and a second partition 154 in such a manner that the first partition 152 is hermetically sealed from the second partition 154. The frame 140 can be integrally formed with the peripheral wall 136, the first sidewall 132, and/or the second sidewall 134. In some examples, the frame 140 is welded to the peripheral wall 136 or welded to the first sidewall 132, and/or the second sidewall 134. The first sidewall 132, the peripheral wall 136, and the frame 140 may form the first partition 152. The second sidewall 134, the peripheral wall 136, and the frame 140 may form the second partition 154, which is opposite the frame 140 from the first partition 152.

The electronic pump device 106 can include a header 126 attached to the housing 120 to form an internal region 158 between an inner surface of the header 126 and an outer surface of the housing 120 that includes power and communication interface structures such as a secondary coil 128 and the antenna 130 external to the hermetically sealed housing 120. The header 126 is configured from a dielectric or insulative material, such as a radome, to allow the transmission of power and communication signals between the antenna 130 and a handset programmer or charger, and between the secondary coil 128 and the charger. For example, the header 126 may include an over-molded polymer affixed to the housing 120 and including the secondary coil 128 and the antenna 130 within the internal region 158. The secondary coil 128 and antenna 130 are constructed from a biocompatible material. In some examples, the secondary coil 128 and antenna 130 can be formed as a coil from a stamped titanium core clad with gold or silver. In some examples, the secondary coil 128 and antenna 130 can be formed from a gold wire.

The electronic pump device 106 includes an energy storage system, such as a battery (e.g., a rechargeable power source) (e.g., a rechargeable battery), and electronic components 112 within the first partition 152. The electronic components 112 can be disposed on a circuit substrate 110, such as a plurality of circuit boards, within the first partition 152. The battery 160 can assume various forms appropriate to provide power for generating desired electrical signals and to store power provided from the electronic components 112. For example, the battery 160 can incorporate lithium-ion (Li+) chemistry, e.g., a lithium-ion battery to operate the electronic components 112. In some examples, the electronic components 112 can be implemented by various components including resistors, capacitors, transistors, and integrated circuits disposed on the circuit substrate 110. The secondary coil 128 and antenna 130 are electrically coupled to the electronic components 112 within the first partition 152, such as via a hermetic feedthrough component.

The electronic components 112 can include a recharge system, a communication system, and a controller. The recharge system includes hardware configured to interface with the secondary coil 128 to receive power signals, and to provide the power signals in a form suitable to recharge the battery 160 and can include circuitry to reduce the likelihood of overcharging the battery 160. The communication system includes hardware configured to interface with the antenna 130 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external devices 101 (e.g., handset programmers). The communication system can include an integrated circuit to implement an applied communication technology. In some examples, the communication system can be used to transmit communication signals to other devices, such as a charger or the handheld programmer (e.g., external device 101), and the communication system can be implemented to generate communication signals and provide the communication signals to the antenna 130 for transmission. In some examples, the communication system can be configured to receive and transmit radio frequency signals via the antenna 130. The controller can include a microcontroller to operate the recharge system and to receive and operate in response to communication signals or generate communication signals from the communication system.

The electronic pump device 106 also includes a fluidic circuit 170 within the second partition 154 and opposite the frame 140 from the battery 160 and electronic components 112. In some examples, the frame 140 can include an opening 142 that includes a hermetic interface 144, such as a feedthrough hermetically affixed to the frame 140. The electronic components 112 are operably coupled to the fluidic circuit 170 across the frame 140 via the hermetic interface 144. For example, the controller of the electronic components 112, powered by the battery 160, can cause the operation of the fluidic circuit 170 such as to control and monitor the fluidic circuit 170.

The fluidic circuit 170 includes a fluidic manifold 108 and fluidic components 174 operably coupled to the fluidic manifold 108. In some examples, the fluidic manifold 108 is a structure integrated into the frame 140 such that the fluidic manifold 108 and the frame 140 together form the hermetic barrier between the first partition 152 and the second partition 154 of the internal compartment 150. For instance, the battery 160, the circuit substrate 110, or electronic components 112 can be coupled to a first major surface of the fluidic manifold 108 in the first partition 152, and the fluidic components 174 are operably coupled to a second, and opposite major surface of the fluidic manifold 108 in the second partition 154.

The fluidic circuit 170 provides for the transfer of the fluid between the fluid reservoir 102 and the inflatable member 104. The fluidic manifold 108, which can be a hermetic manifold, segments and contains the fluid from the internal compartment 150 to reduce the chance of fluid exchange and directs the fluid from a first port 176 to a second port 178 via internal fluid passageways or channels.

The fluidic components 174 include a plurality of fluid pumps, such as pumps 180, 182, a valve 184 mounted into the fluidic manifold 108 in fluidic communication with a manifold passageway to transfer fluid from the first port 176 to the second port 178. The pumps and the valve(s) are in fluid communication with a single fluid passageway between ports 176, 178. The fluidic components 174 also includes one or more pressure sensors 186 operably coupled to the fluidic manifold 108 and in fluidic communication with the passageway to detect a pressure of the fluid within the fluidic manifold 108.

The fluidic components 174 are included in a planar configuration on the fluidic manifold 108 in which the pumps 180, 182, valve 184, and pressure sensor 186 are mounted into the fluidic manifold 108 on a plane for slim profile within the second partition 154. The fluidic manifold 108 can include chambers 188 formed into the second major surface in which the chambers are fluidically coupled to the single passageway within the fluidic manifold 108. The chambers are configured to receive the pumps 180, 182, and valve 184 and one or more pressure sensors 186. In some examples, the fluidic manifold 108 can receive a piezoelectric pump. The fluidic manifold 108 can receive a component cover 190 over the fluidic components 174, which can be hermetically sealed to the second major surface.

In some examples, the electronic pump device 106 may include kink resistant tubing 192 that can extend through the header 126 and attached to the ports 176, 178 via components such as a barb 194 and O-rings. The kink resistant tubing 192 can be attached to the tube members 103, 105 to fluidically couple the electronic pump device 106 to the fluid reservoir 102 and the inflatable member 104.

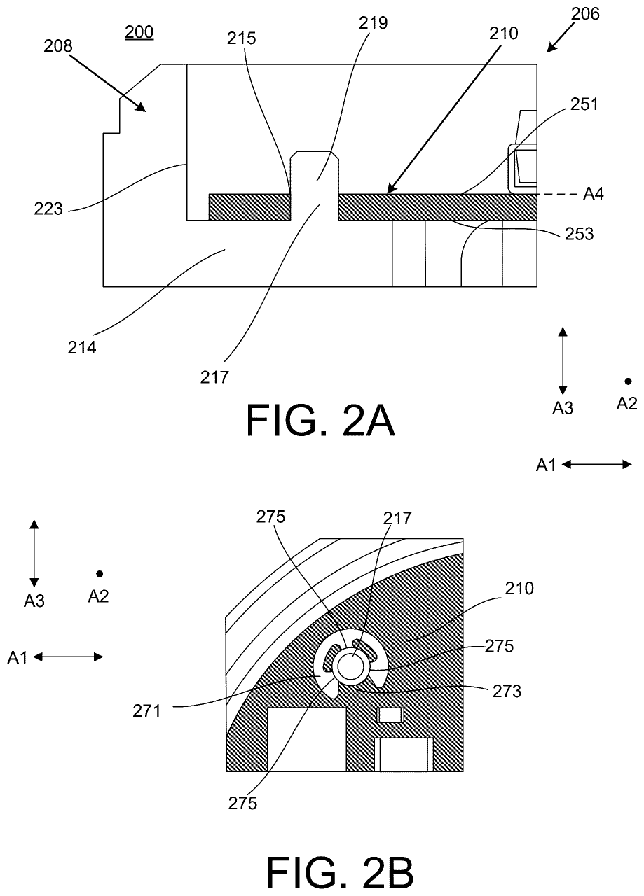

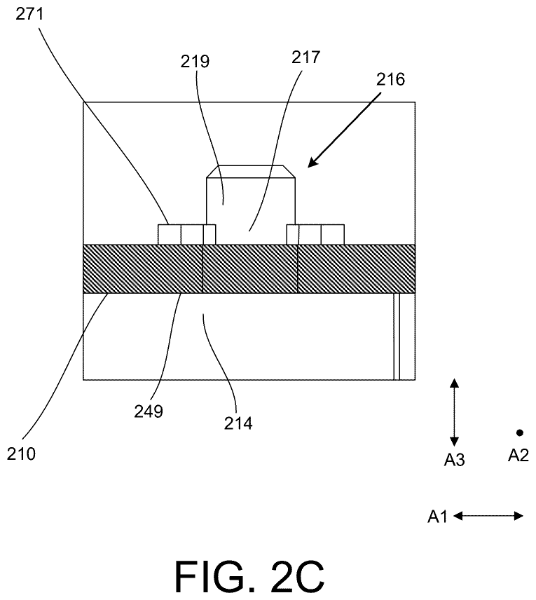

FIGS. 2A to 2C illustrate an aspect of an implantable medical device 200 having a circuit substrate 210 coupled to a shelf 214 of a fluidic manifold 208 using a coupling member 216 according to an aspect. The implantable medical device 200 may be an example of the implantable medical device 100 of FIGS. 1A to 1F and may include any of the details discussed with reference to those figures.

The shelf 214 may extend from the inside edge 223 of a frame (e.g., the frame 140 of FIGS. 1A to 1F). The circuit substrate 210 contacts the shelf 214. The circuit substrate 210 may sit on top of the shelf 214 such that the circuit substrate 210 is positioned within the frame. The circuit substrate 210 includes a first surface 251 and a second surface 253 that is opposite to the first surface 251. The first surface 251 is disposed in a plane A4. The thickness of the circuit substrate 210 is defined as the distance between the first surface 251 and the second surface 253 in the direction A3. The direction A3 is orthogonal to the plane A4. The direction A1 is perpendicular to the direction A3 and perpendicular to the direction A2. The direction A2 is perpendicular to the direction A1 and perpendicular to the direction A3. The shelf 214 extends from the inside edge 223 in the direction A1.

The circuit substrate 210 includes one or more holes 215. In some examples, the holes 215 extend entirely through the thickness of the circuit substrate 210 in the direction A3. The electronic pump device 206 includes one or more coupling members 216 that couples the circuit substrate 210 to the shelf 214. A coupling member 216 may be a component that can extend into a hole 215. As shown in FIGS. 2A to 2C, the coupling member 216 includes a post 217 that is integral to and extends from the shelf 214 in the direction A3. In some examples, the post 217 includes a cylindrical post defining a diameter. In some examples, the post's diameter is less than the diameter of the hole 215. The post 217 extends through and out of the hole 215. The post 217 has a length in the direction A3 that is greater than the thickness of the circuit substrate 210. The post 217 includes a portion 219 that extends out of the hole 215 (e.g., the portion 219 is disposed above the first surface 251 in the direction A3).

The coupling member 216 includes a clip 271 coupled to the post 217, where the circuit substrate 210 has a portion 249 disposed between the clip 271 and the shelf 214. In some examples, the clip 271 is a circular disc with a groove 273. In some examples, the clip 271 includes prongs 275 that contact the post 217.

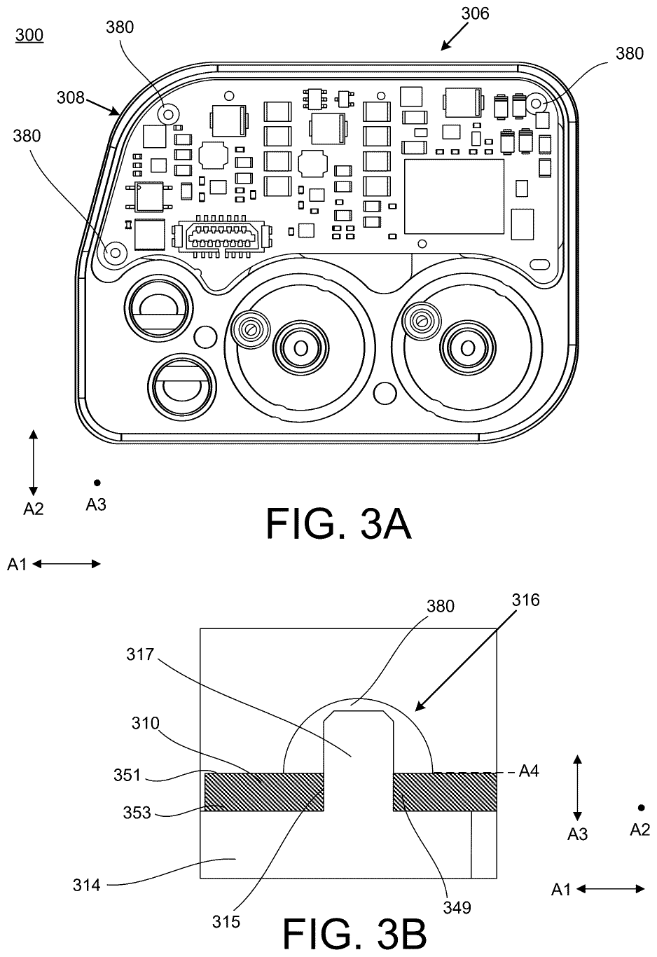

FIGS. 3A and 3B illustrate an aspect of an implantable medical device 300 having a circuit substrate 310 coupled to a shelf 314 of a fluidic manifold 308 using a coupling member 316 according to an aspect. The coupling member 316 includes a post 317 and an adhesive material 380 disposed on the post 317. The implantable medical device 300 may be an example of the implantable medical device 100 of FIGS. 1A to 1F and may include any of the details discussed with reference to those figures.

The shelf 314 may extend from an inside edge (e.g., the inside edge 123 of FIGS. 1A to 1F) of a frame (e.g., the frame 140 of FIGS. 1A to 1F). The circuit substrate 310 contacts the shelf 314. The circuit substrate 310 may sit on top of the shelf 314 such that the circuit substrate 310 is positioned within the frame. The circuit substrate 310 includes a first surface 351 and a second surface 353 that is opposite to the first surface 351. The first surface 351 is disposed in a plane A4. The thickness of the circuit substrate 310 is defined as the distance between the first surface 351 and the second surface 353 in the direction A3. The direction A3 is orthogonal to the plane A4. The direction A1 is perpendicular to the direction A3 and perpendicular to the direction A2. The direction A2 is perpendicular to the direction A1 and perpendicular to the direction A3. The shelf 314 extends from the inside edge in the direction A1.

The circuit substrate 310 includes one or more holes 315. In some examples, the holes 315 extend entirely through the thickness of the circuit substrate 310 in the direction A3. The electronic pump device 306 includes one or more coupling members 316 that couples the circuit substrate 310 to the shelf 314. A coupling member 316 may be a component that can extend into a hole 315. As shown in FIGS. 3A and 3B, the coupling member 316 includes a post 317 that is integral to and extends from the shelf 314 in the direction A3. In some examples, the post 317 includes a cylindrical post defining a diameter. In some examples, the post's diameter is less than the diameter of the hole 315. The post 317 extends through and out of the hole 315. The post 317 has a length in the direction A3 that is greater than the thickness of the circuit substrate 310.

The coupling member 316 includes an adhesive material 380 disposed on the post 317. The circuit substrate 310 has a portion 349 disposed between the adhesive material 380 and the shelf 314. The adhesive material 380 may be an encapsulation (e.g., a high-pressure injection molded material), glue, epoxy, or other adhesive can be dispensed or molded over the top of the post 317. The adhesive material 380 may adhere to both the post 217 and the circuit substrate 310, which may prevent (or reduce) motion in the A1, A2, and/or A3 directions.

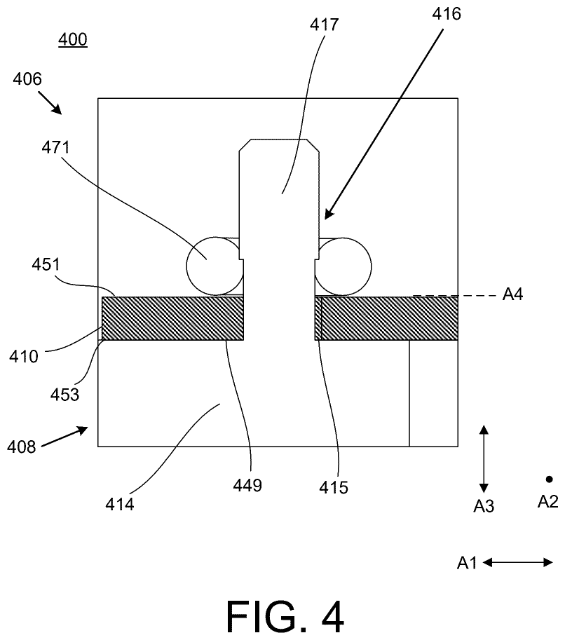

FIG. 4 illustrates an aspect of an implantable medical device 400 having a circuit substrate 410 coupled to a shelf 414 of a fluidic manifold 408 using a coupling member 416 according to an aspect. The implantable medical device 400 may be an example of the implantable medical device 100 of FIGS. 1A to 1F and may include any of the details discussed with reference to those figures.

The shelf 414 may extend from an inside edge (e.g., the inside edge 123 of FIGS. 1A to 1F) of a frame (e.g., the frame 140 of FIGS. 1A to 1F). The circuit substrate 410 contacts the shelf 414. The circuit substrate 410 may sit on top of the shelf 414 such that the circuit substrate 410 is positioned within the frame. The circuit substrate 410 includes a first surface 451 and a second surface 453 that is opposite to the first surface 451. The first surface 451 is disposed in a plane A4. The thickness of the circuit substrate 410 is defined as the distance between the first surface 451 and the second surface 453 in the direction A3. The direction A3 is orthogonal to the plane A4. The direction A1 is perpendicular to the direction A3 and perpendicular to the direction A2. The direction A2 is perpendicular to the direction A1 and perpendicular to the direction A3. The shelf 414 extends from the inside edge in the direction A1.

The circuit substrate 410 includes one or more holes 415. In some examples, the holes 415 extend entirely through the thickness of the circuit substrate 410 in the direction A3. The electronic pump device 406 includes one or more coupling members 416 that couples the circuit substrate 410 to the shelf 414. A coupling member 416 may be a component that can extend into a hole 415. As shown in FIG. 4, the coupling member 416 includes a post 417 that is integral to and extends from the shelf 414 in the direction A3. In some examples, the post 417 includes a cylindrical post defining a diameter. In some examples, the post's diameter is less than the diameter of the hole 415. The post 417 extends through and out of the hole 415. The post 417 has a length in the direction A3 that is greater than the thickness of the circuit substrate 410. The coupling member 416 includes a ring member 471 coupled to the post 417, where the circuit substrate 410 has a portion 449 disposed between the ring member 471 and the shelf 414. In some examples, the ring member 471 is an O-ring.

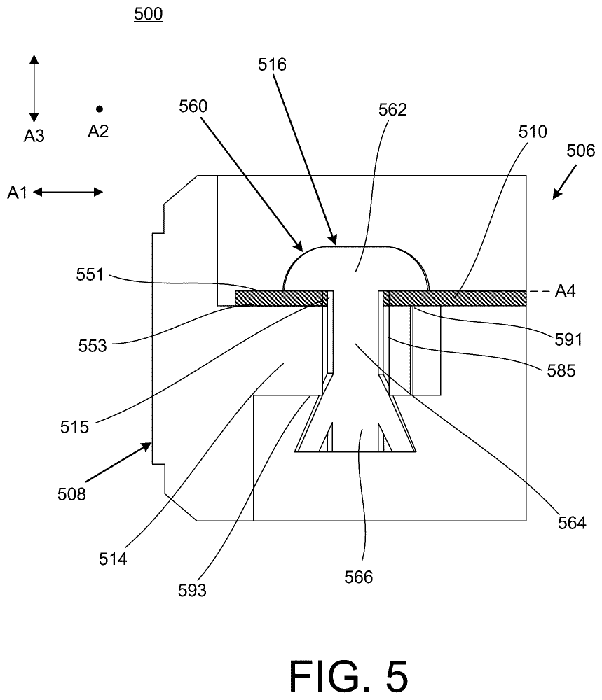

FIG. 5 illustrates an aspect of an implantable medical device 500 having a circuit substrate 510 coupled to a shelf 514 of a fluidic manifold 508 using a coupling member 516 according to an aspect. The implantable medical device 500 may be an example of the implantable medical device 100 of FIGS. 1A to 1F and may include any of the details discussed with reference to those figures.

The shelf 514 may extend from an inside edge (e.g., the inside edge 123 of FIGS. 1A to 1F) of a frame (e.g., the frame 140 of FIGS. 1A to 1F). The circuit substrate 510 contacts the shelf 514. The shelf 514 includes a first surface 591 and a second surface 593. The second surface 593 may be parallel to the first surface 591. The distance between the first surface 591 and the second surface 593 defines the thickness of the shelf 514 in the direction A3. The circuit substrate 510 may sit on top of the shelf 514 such that the circuit substrate 510 is positioned within the frame. The circuit substrate 510 includes a first surface 551 and a second surface 553 that is opposite to the first surface 551. The first surface 551 is disposed in a plane A4. The thickness of the circuit substrate 510 is defined as the distance between the first surface 551 and the second surface 553 in the direction A3. The direction A3 is orthogonal to the plane A4. The direction A1 is perpendicular to the direction A3 and perpendicular to the direction A2. The direction A2 is perpendicular to the direction A1 and perpendicular to the direction A3. The shelf 514 extends from the inside edge in the direction A1.

The circuit substrate 510 includes one or more holes 515. In some examples, the holes 515 extend entirely through the thickness of the circuit substrate 510 in the direction A3. The shelf 514 includes one or more holes 585. The hole(s) 585 may extend entirely through the shelf 514. The hole(s) 585 may be aligned with the hole(s) 515 in the direction A3. In some examples, the hole(s) 515 and the hole(s) 585 may have the same size (e.g., same diameter). The electronic pump device 506 includes one or more coupling members 516 that couples the circuit substrate 510 to the shelf 514. A coupling member 516 may be a component that can extend through hole 515 and hole 585.

As shown in FIG. 5, the coupling member 516 includes a rivet 560. The rivet 560 may be positioned in the hole 515 and the hole 585. The rivet 560 includes one or more features that contact the circuit substrate 510 and the shelf 514 to hold the circuit substrate 510 and the shelf 514 together. The rivet 560 includes a head portion 562, a neck portion 564, and a flared portion 566. The neck portion 564 may have a size (e.g., a diameter) that is smaller than the size (e.g., diameter) of the hole(s) 515 and the hole(s) 585. In some examples, the head portion 562 includes a dome shape. The head portion 562 has a size that is larger than the size of the hole(s) 515 and the hole(s) 585. The head portion 562 contacts the first surface 551 of the circuit substrate 510. The flared portion 566 contacts portions (e.g., the corners defined by the intersection of the second surface 593 and the hole 585) of the shelf 514. The flared portion 566 may be a portion that increases in width (e.g., in the direction A1) along its length in the direction A3. In some examples, the rivet 560 is assembled from the top side of the circuit substrate 510, and the rivet 560 is deployed through the hole 515 in the circuit substrate 510 and the hole 585 in the shelf 514. The deployed rivet 560 may contact the bottom of the hole 585 in the shelf 514, which may lock the circuit substrate 510 in place.

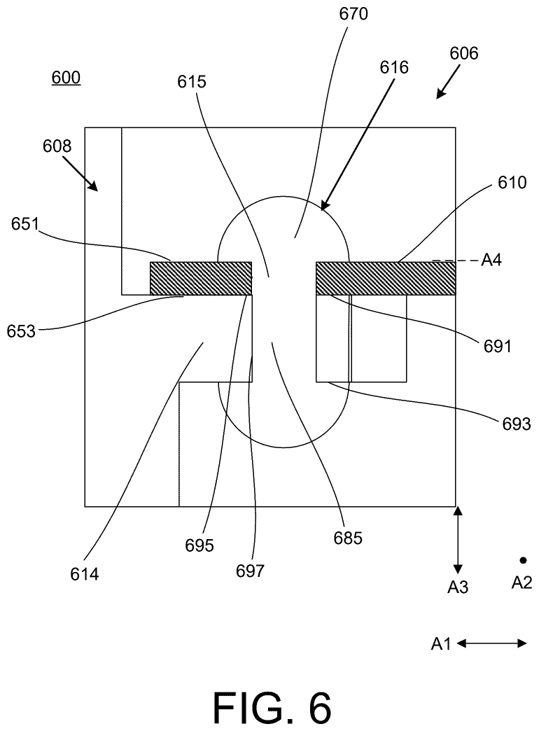

FIG. 6 illustrates an aspect of an implantable medical device 600 having a circuit substrate 610 coupled to a shelf 614 of a fluidic manifold 608 using a coupling member 516 according to an aspect. The implantable medical device 600 may be an example of the implantable medical device 100 of FIGS. 1A to 1F and may include any of the details discussed with reference to those figures.

The shelf 614 may extend from an inside edge (e.g., the inside edge 123 of FIGS. 1A to 1F) of a frame (e.g., the frame 140 of FIGS. 1A to 1F). The circuit substrate 610 contacts the shelf 614. The shelf 614 includes a first surface 691 and a second surface 693. The second surface 693 may be parallel to the first surface 691. The distance between the first surface 691 and the second surface 693 defines the thickness of the shelf 614 in the direction A3. The circuit substrate 610 may sit on top of the shelf 614 such that the circuit substrate 610 is positioned within the frame. The circuit substrate 610 includes a first surface 651 and a second surface 653 that is opposite to the first surface 651. The first surface 651 is disposed in a plane A4. The thickness of the circuit substrate 610 is defined as the distance between the first surface 651 and the second surface 653 in the direction A3. The direction A3 is orthogonal to the plane A4. The direction A1 is perpendicular to the direction A3 and perpendicular to the direction A2. The direction A2 is perpendicular to the direction A1 and perpendicular to the direction A3. The shelf 614 extends from the inside edge in the direction A1.

The circuit substrate 610 includes one or more holes 615. In some examples, the holes 615 extend entirely through the thickness of the circuit substrate 610 in the direction A3. The shelf 614 includes one or more holes 685. The hole(s) 685 may extend entirely through the shelf 614. The hole(s) 685 may be aligned with the hole(s) 615 in the direction A3. In some examples, the hole(s) 615 and the hole(s) 685 may have the same size (e.g., same diameter). The electronic pump device 606 includes one or more coupling members 616 that couples the circuit substrate 610 to the shelf 614. A coupling member 616 may be a component that can extend through hole 615 and hole 685.

As shown in FIG. 6, the coupling member 616 includes injection molding 670. The injection molding 670 is positioned on a portion of the circuit substrate 610, in the hole 615, in the hole 685, and on a portion of the shelf 614. The injection molding 670 may form an anchor that is disposed within the holes 615, 685, and on portions of the circuit substrate 610 and the shelf 614. The injection molding 670 may form a cap feature (e.g., a dome portion) on top of the circuit substrate 610 and a cap feature (e.g., a dome portion) on the bottom of the shelf 614. The injection molding 670 may contact edges 695 of the circuit substrate 610 that form the hole 615 and may contact the first surface 651 of the circuit substrate 610. The injection molding 670 may contact edges 697 of the shelf 614 that form the hole 685 and may contact the second surface 693 of the shelf 614.

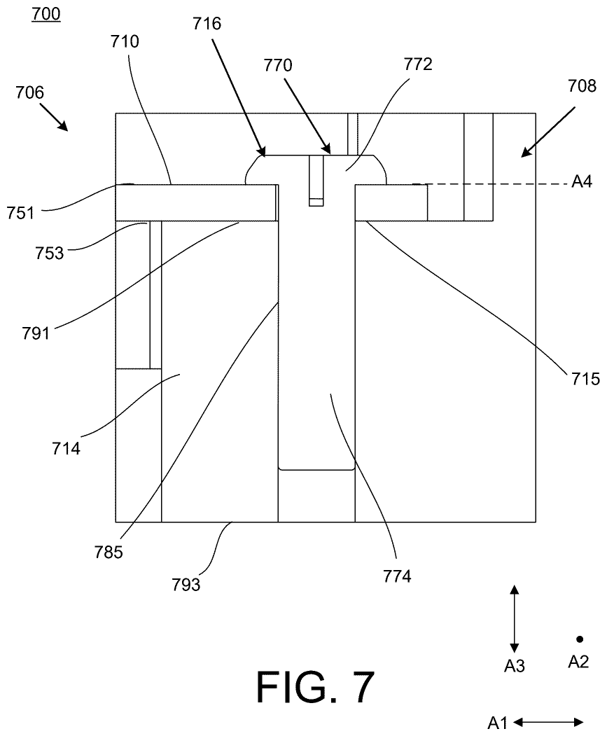

FIG. 7 illustrates an aspect of an implantable medical device 700 having a circuit substrate 710 coupled to a shelf 714 of a fluidic manifold 708 using a coupling member 716 according to an aspect. The implantable medical device 700 may be an example of the implantable medical device 100 of FIGS. 1A to 1F and may include any of the details discussed with reference to those figures.

The shelf 714 may extend from an inside edge (e.g., the inside edge 123 of FIGS. 1A to 1F) of a frame (e.g., the frame 140 of FIGS. 1A to 1F). The circuit substrate 710 contacts the shelf 714. The shelf 714 includes a first surface 791 and a second surface 793. The second surface 793 may be parallel to the first surface 791. The distance between the first surface 791 and the second surface 793 defines the thickness of the shelf 714 in the direction A3. The circuit substrate 710 may sit on top of the shelf 714 such that the circuit substrate 710 is positioned within the frame. The circuit substrate 710 includes a first surface 751 and a second surface 753 that is opposite to the first surface 751. The first surface 751 is disposed in a plane A4. The thickness of the circuit substrate 710 is defined as the distance between the first surface 751 and the second surface 753 in the direction A3. The direction A3 is orthogonal to the plane A4. The direction A1 is perpendicular to the direction A3 and perpendicular to the direction A2. The direction A2 is perpendicular to the direction A1 and perpendicular to the direction A3. The shelf 714 extends from the inside edge in the direction A1.

The circuit substrate 710 includes one or more holes 715. In some examples, the holes 715 extend entirely through the thickness of the circuit substrate 710 in the direction A3. The shelf 714 includes one or more holes 785. The hole(s) 785 may extend entirely through the shelf 714. In some examples, the hole(s) 785 may extend partially through the shelf 714. The hole(s) 785 may be aligned with the hole(s) 715 in the direction A3. In some examples, the hole(s) 715 and the hole(s) 785 may have the same size (e.g., same diameter). In some examples, the hole(s) 715 are larger than the hole(s) 785. In some examples, the hole(s) 785 are threaded holes. In some examples, the hole(s) 715 are non-threaded holes, e.g., through-holes. The electronic pump device 706 includes one or more coupling members 716 that couples the circuit substrate 710 to the shelf 714. A coupling member 716 may be a component that can extend through hole 715 and hole 785.

As shown in FIG. 7, the coupling member 716 includes a threaded fastener 770. The threaded fastener 770 includes a head portion 772 and an elongated member 774. The head portion 772 contacts the first surface 751 of the circuit substrate 710. The elongated member 774 defines the threads that interact with the threads of the hole 785. In some examples, the threaded fastener 770 is loaded from the top side of the circuit substrate 710. In some examples, the hole 715 in the circuit substrate 710 is slightly larger than the thread diameter allowing the threaded fastener 770 to pass through the circuit substrate 710 and then engage with the threads on the shelf 714.

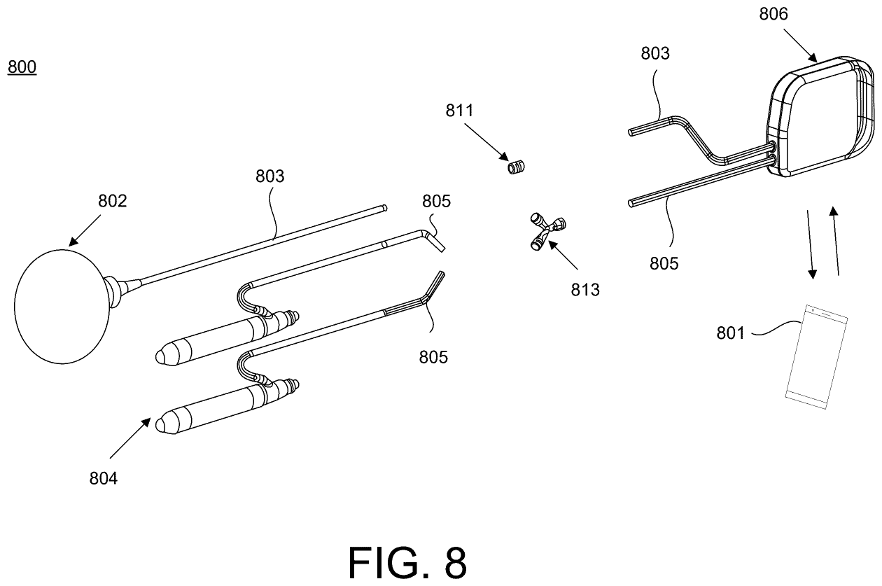

FIG. 8 illustrates a perspective of an inflatable penile prosthesis 800 according to an aspect. The inflatable penile prosthesis 800 may be an example of any of the medical devices discussed herein (e.g., including implantable medical device 100), and, therefore, may include any of the details discussed with reference to the previous figures.

The inflatable penile prosthesis 800 includes an inflatable member 804, a fluid reservoir 802, and an electronic pump device 806. The inflatable member 804 includes a pair of inflatable cylinders. The electronic pump device 806 may be an example of any of the pump devices discussed with reference to the previous figures and may include any of the details discussed herein. The electronic pump device 806 includes fluidic components such as pumps, valves, and/or sensing devices positioned in fluid passageways. The electronic pump device 806 includes components such as, for example, one or more fluid control devices, one or more pressure sensors, and other such components. The electronic pump device 806 includes an electronic control system configured to provide for the transfer of fluid between a fluid reservoir 802 and an inflatable member 804 via the fluidic components.

The electronic pump device 806 may include one or more integrated circuits. In some examples, the integrated circuits are included in a printed circuit board that is included in a housing of the electronic pump device 806. Fluidic components and the electronic components of the electronic pump device 806 are included in a housing. In some examples, fluidic components and electronic components in the housing define a manifold (e.g., an electronically controlled fluidic manifold) that provides for the electronic control of the flow of fluid between the fluid reservoir 802 and the inflatable member 804. In some examples, the electronic pump device 806 can communicate with an external device 801, via respective communication modules. For example, an application stored in a memory and executed by a processor of the external device 801 may allow the user and/or a physician to operate, view, monitor and alter operation of the inflatable penile prosthesis 800.

The inflatable penile prosthesis 800 includes one or more first tube members 803 that connect a first fluid port of the electronic pump device 806 with the fluid reservoir 802. One or more second tube members 805 connect a second fluid port of the electronic pump device 806 with the inflatable member 804 in the form of the inflatable cylinders. In some examples, the inflatable penile prosthesis 800 includes a connector 811 that is used to connect two tube members 803 together, and a connector 813 that is used to connect two tube members 805 together.

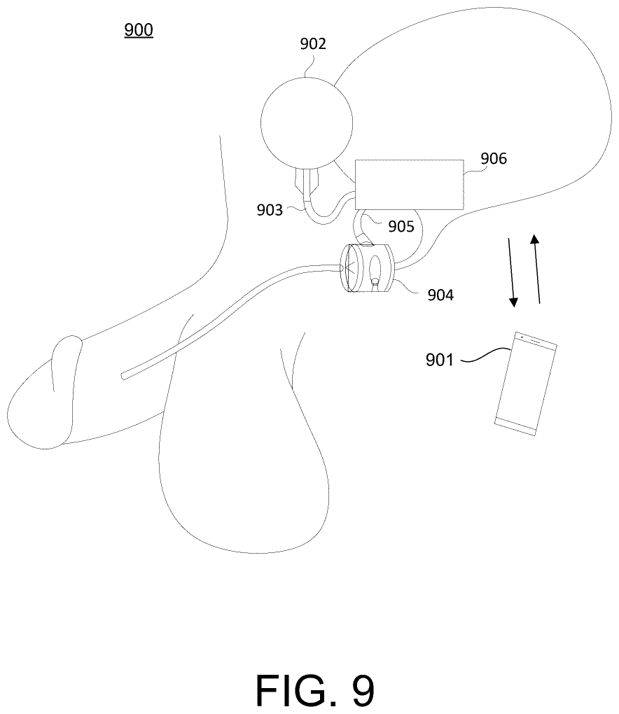

FIG. 9 illustrates a urinary control device 900 having an electronic pump device 906 according to an aspect. The urinary control device 900 may be an example of the implantable medical device 100. In some examples, the urinary control device 900 is an artificial urinary sphincter device. The electronic pump device 906 may include any of the features of the pump devices discussed herein. The urinary control device 900 includes an electronic pump device 906, a fluid reservoir 902, and a cuff 904 (e.g., an inflatable cuff).

The fluid reservoir 902 may be a pressure-regulating inflation balloon or element. The fluid reservoir 902 is in operative fluid communication with the cuff 904 via one or more tube members 903, 905. The fluid reservoir 902 is constructed of polymer material that is capable of elastic deformation to reduce fluid volume within the fluid reservoir 902 and push fluid out of the fluid reservoir 902 and into the cuff 904. However, the material of the fluid reservoir 902 can be biased or include a shape memory construct adapted to generally maintain the fluid reservoir 902 in its expanded state with a relatively constant fluid volume and pressure. In some examples, this constant level of pressure exerted from the fluid reservoir 902 to the cuff 904 will keep the cuff 904 at a desired inflated state when open fluid communication is provided between the fluid reservoir 902 and the cuff 904. In some examples, the fluid reservoir 902 is implanted into the abdominal space.

A user may use an external device 901 to control the urinary control device 900. In some examples, the user may use the external device 901 to inflate or deflate the cuff 904. For example, in response to the user activating an inflation cycle using the external device 901, the external device 901 may transmit a wireless signal to the electronic pump device 906 to initiate the inflation cycle to transfer fluid from the fluid reservoir 902 to the cuff 904 (e.g., by opening an active valve where the pressure in the fluid reservoir 902 causes the fluid to move through the active valve to the cuff 904). In some examples, in response to the user activating a deflation cycle using the external device 901, the external device 901 may transmit a wireless signal to the pump device 906 to initiate the deflation cycle to transfer fluid from the cuff 904 to the fluid reservoir 902.

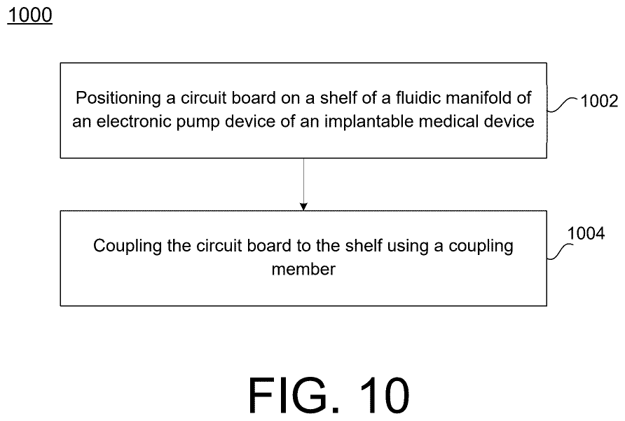

FIG. 10 illustrates a flowchart 1000 depicting example operations of coupling a circuit substrate to a fluidic manifold of an electronic pump device according to an aspect. Although the flowchart 1000 of FIG. 10 illustrates the operations in sequential order, it will be appreciated that this is merely an example, and that additional or alternative operations may be included. Further, operations of FIG. 10 and related operations may be executed in a different order than that shown, or in a parallel or overlapping fashion.

Operation 1002 includes positioning a circuit board on a shelf of a fluidic manifold of an electronic pump device of an implantable medical device. Operation 1004 includes coupling the circuit board to the shelf using a coupling member.

Clause 1. An implantable medical device comprising: an inflatable member; a fluid reservoir; and an electronic pump device configured to transfer fluid between the inflatable member and the fluid reservoir, the electronic pump device including a housing, the housing including: a fluidic manifold including a plurality of fluidic components, the manifold including an inside edge and a shelf that extends from the inside edge; a circuit substrate contacting the shelf, the circuit substrate including a hole; and a coupling member that couples the circuit substrate to the shelf, the coupling member extending through the hole.

Clause 2. The implantable medical device of clause 1, wherein the coupling member includes a post that is integral to and extends from the shelf.

Clause 3. The implantable medical device of clause 2, wherein the coupling member includes a clip coupled to the post, the circuit substrate having a portion disposed between the clip and the shelf.

Clause 4. The implantable medical device of clause 2, wherein the coupling member includes an adhesive material disposed on the post.

Clause 5. The implantable medical device of clause 2, wherein the coupling member includes a ring member coupled to the post, the circuit substrate having a portion disposed between the ring member and the shelf.

Clause 6. The implantable medical device of clause 1, wherein the coupling member includes a rivet.

Clause 7. The implantable medical device of clause 6, wherein the hole is a first hole, the shelf including a second hole, the rivet includes a head portion, a neck portion, and a flared portion, the head portion contacting the circuit substrate, the neck portion being disposed in the first hole and the second hole, the flared portion connecting the shelf.

Clause 8. The implantable medical device of clause 1, wherein the coupling member includes injection molding.

Clause 9. The implantable medical device of clause 1, wherein the coupling member includes a threaded fastener.

Clause 10. The implantable medical device of clause 9, wherein the hole is a first hole, the shelf including a second hole, the second hole including threads, the threaded fastener including a head portion and an elongated member with threads, the head portion contacting the circuit substrate, a portion of the elongated member being disposed in the second hole.

Clause 11. The implantable medical device of any one of clauses 1 to 10, wherein the implantable medical device includes a penile prosthesis, and the inflatable member includes a pair of cylinders.

Clause 12. The implantable medical device of any one of clauses 1 to 10, wherein the implantable medical device includes a urinary control device, and the inflatable member includes an inflatable cuff.

Clause 13. A method comprising: positioning a circuit board on a shelf of a fluidic manifold of an electronic pump device of an implantable medical device; and coupling the circuit board to the shelf using a coupling member.

Clause 14. The method of clause 13, wherein the coupling member includes a post that is integral to and extends from the shelf, the coupling member including an adhesive material disposed on the post, a clip coupled to the post, or a ring member coupled to the post.

Clause 15. The method of clause 13, wherein the coupling member includes a rivet, injection molding, or a threaded fastener.

Clause 16. An implantable medical device comprising: an inflatable member; a fluid reservoir; and an electronic pump device configured to transfer fluid between the inflatable member and the fluid reservoir, the electronic pump device including a housing, the housing including: a fluidic manifold including a plurality of fluidic components, the manifold including an inside edge and a shelf that extends from the inside edge; a circuit substrate contacting the shelf, the circuit substrate including a hole; and a coupling member that couples the circuit substrate to the shelf, the coupling member extending through the hole.

Clause 17. The implantable medical device of clause 16, wherein the coupling member includes a post that is integral to and extends from the shelf.

Clause 18. The implantable medical device of clause 17, wherein the coupling member includes a clip coupled to the post, the circuit substrate having a portion disposed between the clip and the shelf.

Clause 19. The implantable medical device of clause 17, wherein the coupling member includes an adhesive material disposed on the post.

Clause 20. The implantable medical device of clause 17, wherein the coupling member includes a ring member coupled to the post, the circuit substrate having a portion disposed between the ring member and the shelf.

Clause 21. The implantable medical device of clause 16, wherein the coupling member includes a rivet.

Clause 22. The implantable medical device of clause 21, wherein the hole is a first hole, the shelf including a second hole, the rivet includes a head portion, a neck portion, and a flared portion, the head portion contacting the circuit substrate, the neck portion being disposed in the first hole and the second hole, the flared portion connecting the shelf.

Clause 23. The implantable medical device of clause 16, wherein the coupling member includes injection molding.

Clause 24. The implantable medical device of clause 16, wherein the coupling member includes a threaded fastener.

Clause 25. The implantable medical device of clause 24, wherein the hole is a first hole, the shelf including a second hole, the second hole including threads, the threaded fastener including a head portion and an elongated member with threads, the head portion contacting the circuit substrate, a portion of the elongated member being disposed in the second hole.

Clause 26. The implantable medical device of clause 16, wherein the implantable medical device includes a penile prosthesis, and the inflatable member includes a pair of cylinders.

Clause 27. The implantable medical device of clause 16, wherein the implantable medical device includes a urinary control device, and the inflatable member includes an inflatable cuff.

Clause 28. A method comprising: positioning a circuit board on a shelf of a fluidic manifold of an electronic pump device of an implantable medical device; and coupling the circuit board to the shelf using a coupling member.

Clause 29. The method of clause 28, wherein the coupling member includes a post that is integral to and extends from the shelf, the coupling member including an adhesive material disposed on the post, a clip coupled to the post, or a ring member coupled to the post.

Clause 30. The method of clause 28, wherein the coupling member includes a rivet, injection molding, or a threaded fastener.

Clause 31. An implantable medical device comprising: an inflatable member; a fluid reservoir; and an electronic pump device configured to transfer fluid between the inflatable member and the fluid reservoir, the electronic pump device including a housing, the housing including: a fluidic manifold including one or more pumps, one or more valves, or one or more pressure sensors, the fluidic manifold including a frame with an inside edge, the fluidic manifold including a shelf that extends from the inside edge; a circuit substrate contacting the shelf, the circuit substrate including a plurality of electronic components, the circuit substrate including a hole; and a coupling member that couples the circuit substrate to the shelf, the coupling member extending through the hole.

Clause 32. The implantable medical device of clause 31, wherein the coupling member includes a post that is integral to and extends from the shelf, wherein the coupling member includes an adhesive material disposed on the post, a clip coupled to the post, or a ring member coupled to the post.

Clause 33. The implantable medical device of clause 31, wherein the hole is a first hole, the shelf including a second hole, the second hole being aligned with the first hole, the coupling member having a first portion disposed in the first hole, and a second portion disposed in the second hole, the coupling member including a rivet, injection molding, or a threaded fastener.

Clause 34. The implantable medical device of clause 31, wherein the implantable medical device includes a penile prosthesis, and the inflatable member includes a pair of cylinders.

Clause 35. The implantable medical device of clause 31, wherein the implantable medical device includes a urinary control device, and the inflatable member includes an inflatable cuff.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir; and
an electronic pump device configured to transfer fluid between the inflatable member and the fluid reservoir, the electronic pump device including a housing, the housing including:
a fluidic manifold including a plurality of fluidic components, the manifold including an inside edge and a shelf that extends from the inside edge;
a circuit substrate contacting the shelf, the circuit substrate including a hole; and
a coupling member that couples the circuit substrate to the shelf, the coupling member extending through the hole.

2. The implantable medical device of claim 1, wherein the coupling member includes a post that is integral to and extends from the shelf.

3. The implantable medical device of claim 2, wherein the coupling member includes a clip coupled to the post, the circuit substrate having a portion disposed between the clip and the shelf.

4. The implantable medical device of claim 2, wherein the coupling member includes an adhesive material disposed on the post.

5. The implantable medical device of claim 2, wherein the coupling member includes a ring member coupled to the post, the circuit substrate having a portion disposed between the ring member and the shelf.

6. The implantable medical device of claim 1, wherein the coupling member includes a rivet.

7. The implantable medical device of claim 6, wherein the hole is a first hole, the shelf includes a second hole, the rivet includes a head portion, a neck portion, and a flared portion, the head portion contacts the circuit substrate, the neck portion being disposed in the first hole and the second hole, and the flared portion connects the shelf.

8. The implantable medical device of claim 1, wherein the coupling member includes injection molding.

9. The implantable medical device of claim 1, wherein the coupling member includes a threaded fastener.

10. The implantable medical device of claim 9, wherein the hole is a first hole, the shelf including a second hole, the second hole including threads, the threaded fastener including a head portion and an elongated member with threads, the head portion contacting the circuit substrate, a portion of the elongated member being disposed in the second hole.

11. The implantable medical device of claim 1, wherein the implantable medical device includes a penile prosthesis, and the inflatable member includes a pair of cylinders.

12. The implantable medical device of claim 1, wherein the implantable medical device includes a urinary control device, and the inflatable member includes an inflatable cuff.

13. A method comprising:
positioning a circuit board on a shelf of a fluidic manifold of an electronic pump device of an implantable medical device; and
coupling the circuit board to the shelf using a coupling member.

14. The method of claim 13, wherein the coupling member includes a post that is integral to and extends from the shelf, the coupling member including an adhesive material disposed on the post, a clip coupled to the post, or a ring member coupled to the post.

15. The method of claim 13, wherein the coupling member includes a rivet, injection molding, or a threaded fastener.

16. An implantable medical device comprising:
an inflatable member;
a fluid reservoir; and
an electronic pump device configured to transfer fluid between the inflatable member and the fluid reservoir, the electronic pump device including a housing, the housing including:
a fluidic manifold including one or more pumps, one or more valves, or one or more pressure sensors, the fluidic manifold including a frame with an inside edge, the fluidic manifold including a shelf that extends from the inside edge;
a circuit substrate contacting the shelf, the circuit substrate including a plurality of electronic components, the circuit substrate including a hole; and
a coupling member that couples the circuit substrate to the shelf, the coupling member extending through the hole.

17. The implantable medical device of claim 16, wherein the coupling member includes a post that is integral to and extends from the shelf, wherein the coupling member includes an adhesive material disposed on the post, a clip coupled to the post, or a ring member coupled to the post.

18. The implantable medical device of claim 16, wherein the hole is a first hole, the shelf including a second hole, the second hole being aligned with the first hole, the coupling member having a first portion disposed in the first hole, and a second portion disposed in the second hole, the coupling member including a rivet, injection molding, or a threaded fastener.

19. The implantable medical device of claim 16, wherein the implantable medical device includes a penile prosthesis, and the inflatable member includes a pair of cylinders.

20. The implantable medical device of claim 16, wherein the implantable medical device includes a urinary control device, and the inflatable member includes an inflatable cuff.
